(19) United States
(12) Patent Application Publication
(10) Pub. No.: US 2024/0350258 A1
(43) Pub. Date: Oct. 24, 2024

(54) SPIN-ASSISTED LAYER BY LAYER POLYMER COATINGS FOR INTRAOCULAR LENS (IOL) CARTRIDGES AND A PRODUCTION METHOD THEREOF

(71) Applicant: VSY BIYOTEKNOLOJI VE ILAC SANAYI ANONIM SIRKETI, Istanbul (TR)

(72) Inventor: Faruk OYTUN, Istanbul (TR)

(73) Assignee: VSY BIYOTEKNOLOJI VE ILAC SANAYI ANONIM SIRKETI, Istanbul (TR)

(21) Appl. No.: 18/682,622

(22) PCT Filed: Aug. 10, 2021
(86) PCT No.: PCT/TR2021/050784

§ 371 Date: Feb. 9, 2024

(51) Int. Cl. A61F 2/16; A61L 27/34

(52) U.S. Cl. CPC A61F 2/1675; A61F 2/1678; A61L 27/34; A61L 2400/10; A61L 2420/02; A61L 2420/08; A61L 2430/16

(57) ABSTRACT

Spin-assisted layer-by-layer polymer coatings are used as the inner surface coating material for intraocular lens cartridges and facilitate the implantation of intraocular lenses.

### CROSS REFERENCE TO RELATED APPLICATIONS

This application is a national stage entry of International Application No. PCT/TR2021/050784, filed on Aug. 10, 2021, the entire contents of which are incorporated herein by reference.

### TECHNICAL FIELD

The present invention relates to spin-assisted layer-by-layer (LBL) polymer coatings which are used as the inner surface coating material for the intraocular lens (IOL) cartridges and facilitate the implantation of IOLs.

### BACKGROUND

The natural crystal lens in the human eye loses its transparency over time due to old age, impact or some diseases, and the vision deteriorates with the decrease of light reaching the retina. In this case, intraocular lenses (IOL) are implanted into the eye in place of the natural crystal lens after cataract surgery. An incision is made in the eye during the implantation of intraocular lenses (IOL). This incision should be as small as possible to reduce trauma and accelerate recovery.

In the state of the art, these intraocular lenses (IOL) were made of polymethyl methacrylate (PMMA) material in the early days due to its biocompatible feature Since PMMA is a hard polymer, a 5-7 mm incision was required for its implantation. Since an incision of this size requires suture, it decreases the comfort of the patient and prolongs the healing process. Implantation performed with smaller incision sizes not only eliminates the need for sutures, but also accelerates the healing process of the patient. For this reason, today, acrylic based foldable, flexible intraocular lenses (IOLs) having both hydrophilic and hydrophobic properties are produced. These lenses can be implanted into the eye even in incision sizes of 3 mm or less.

Cartridge injector systems are used in cataract surgeries for implanting the lens into the eye. The lens is folded in the cartridge and passed through the small diameter cartridge tunnel, and then unfolded in the lens capsule located in the eye.

IOL cartridges are generally produced from polymers such as polyolefin (e.g. polypropylene) having high hydrophobic properties. When the IOL is pushed inside the cartridge made of these polymers having high friction force, the movement of the lens inside the cartridge is prevented. As the pressure increases, IOL which is folded inside the cartridge has a tendency to expand inside the cartridge with the effect of the friction force, and it becomes impossible to come out of the end of the cartridge. In this case, implantation fails and the IOL undergoes physical deformations such as rupture, tear, and scratching.

Recently, different methods have been used to minimize the friction in the cartridge and facilitate the implantation of the intraocular lens (IOL) by coming out of the cartridge tip. One of these methods is adding fatty acid esters such as glycerol monostearate (GMS) to the material of the cartridge as a lubricant additive in the production process. The cartridges produced with this method are subjected to high temperatures for the lubricant additive to impregnate into the inner surface of the cartridge. Even though the cartridge to which a lubricant is added provides a very effective slippery coating, these fatty acid esters (lubricant) rise to the surface of the cartridge over time. This lubricant material, which has risen to the surface, may adhere to the surface of the intraocular lens (IOL) during its long shelf life, causing its optical properties to be damaged. Therefore, the shelf life of the cartridges produced by this method should be kept short.

Generally during the application, a viscoelastic gel is added between the lens surface and the coated cartridge surface, allowing the lens and the cartridge to be activated and the lens to slide easily inside the cartridge. For example, important risks and disadvantages such as inability to adjust the viscoelastic fluid used for sliding the lens during surgeries, inability to spread viscoelastic gel homogeneously, strains resulting during the delivery of the lens and scratches and deformations occurring in the lens when low amount of viscoelastic gel is used, as well as difficulty in cleaning after the implantation, the risk of changing the optical properties of the lens by covering the surface of the lens when higher amount of viscoelastic gel is used can be experienced.

Layer-by-layer (LBL) deposition is a very promising approach consisting of sequential immersion of a substrate into solutions of oppositely charged materials. Major advantage of LBL deposition is that it allows one to control the structure of the coatings with actual nanometer scale precision, which includes both normal and lateral packing of the nanoscale building blocks. This method can create versatile thin films with highly tunable thickness, porosity, packing density and surface properties. Furthermore, the multilayer film has excellent adhesion to the substrate since the multilayer is formed via covalent bonding or ionic interaction. Thus, it has been widely utilized for fabrication of solution processed polymer and nanomaterial multilayer films for various applications.

United States patent document no U.S. Pat. No. 8,323,799B2, an application known in the state of the art, discloses that a solution formed by formulating polyvinylpyrrolidone (PVP) or hyaluronic acid (HA) as a hydrophilic lubricant, a commercial urethane dispersion (NeoRez® R-9330) as a matrix polymer and polyfunctional aziridine as a crosslinking agent is used as an IOL cartridge coating material. Within the scope of the said application, it is stated that the cartridges subjected to plasma treatment are coated with the prepared coating solution and then left to dry overnight at 60° C.

Another method known in the art is to apply a polymer-based lubricant film coating on the inner surface of the cartridge. In patent applications U.S. Pat. No. 6,238,799B1 and U.S. Pat. No. 6,866,936B2 made in accordance with this method, mixtures comprising polyacrylates, polymethacrylates, polyurethanes, polyethylene and polypropylene copolymers, polyvinyl chlorides, epoxides, polyamides, polyesters and alkyd copolymers as matrix polymer; poly(N-vinyl lactams), poly(vinylpyrrolidone), poly(ethylene oxide) polypropylene oxide) polyacrylamides, cellulosics, methyl cellulose, polyacrylic acids, polyvinyl alcohols, and polyvinyl ethers as hydrophilic polymers; and at least one crosslink agent are used as IOL coating material. The coating process is performed by applying this mixture to IOL cartridges. The lubricant coatings disclosed in the said applications are relatively hard and non-flexible. This situation has a risk that the cartridge inside the cartridge may detach from the coated surface due to its hard structure and may damage the lens during the implantation process.

United States patent document no U.S. Pat. No. 8,821,572B2, an application known in the state of the art, discloses that IOL coating material comprises polyurethane and PVP which is a hydrophilic polymer and a cross-linking agent Here, it is aimed to apply the coating directly to the inner surface of the cartridge as a single layer. It is considered that this coating applied as a single layer will not be stable during its long shelf-life.

An article titled “Preparation and evaluation of a lubricious treated cartridge used for implantation of intraocular lenses”, one of the applications known in the state of the art, discloses that cartridges subjected to plasma treatment are immersed in a coating solution formed by using polyethylene imine (PEI), PVP as IOL coating material and glutaraldehyde as crosslinking agent and cured at 70° C. It is explained that after the lenses are placed in the coated cartridges, viscoelastic gel is injected before implantation to help the lens slide more easily, and it is waited for 4.5 minutes the lens to be activated. This is quite a long time and carries great risks during the surgical operation. Implantation should be performed in a short time after both viscoelastic gel and saline solution, which will ensure that the lens and coating material are activated, are added.

United States patent document no US20170128195A1, an application known in the state of the art, discloses a solution comprising polyurethane and fluorescing sodium salt as IOL coating material and polyfunctional aziridine which is a crosslinking agent. It is stated that the coated cartridges are exposed to UV light of 254 nm and the indicator properties are observed whether the fluorescent salt showing fluorescent properties is coated homogeneously on the cartridge.

### SUMMARY

The objective of the invention is to obtain spin-assisted layer-by-layer polymer coatings which are used as the inner coating material for the cartridge of IOLs and facilitate the implantation of IOLs.

Another objective of the invention is to obtain a coating that shows successful results in the delivery of silicone-based, hydrophobic acrylic and hydrophilic acrylic based intraocular lenses.

Another object of the invention is to obtain lubricious coatings that provide excellent delivery that can be used in both typical butterfly cartridge injector systems and preloaded injector systems.

Another objective of the invention is to obtain stable coatings during long-term shelf life after sterilization processes and show no transfer of the coating to the IOL during delivery.

Another objective of the invention is to create versatile coatings with highly tunable thickness and surface properties.

Another objective of the invention is to obtain excellent adhesion of the coatings to the IOL cartridge via electrostatic interaction between opposite charged polymer coating materials.

Another object of the invention is to allow IOL implantation from the cartridge with a minimum outlet orifice (3) diameter of 1.0 mm and a maximum outlet orifice (3) diameter of 3.0 mm.

### DETAILED DESCRIPTION OF THE EMBODIMENTS

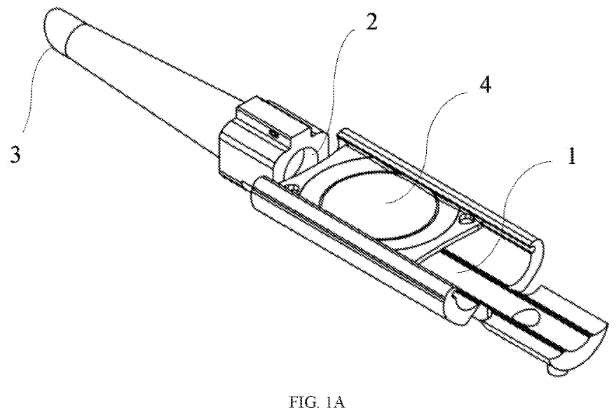

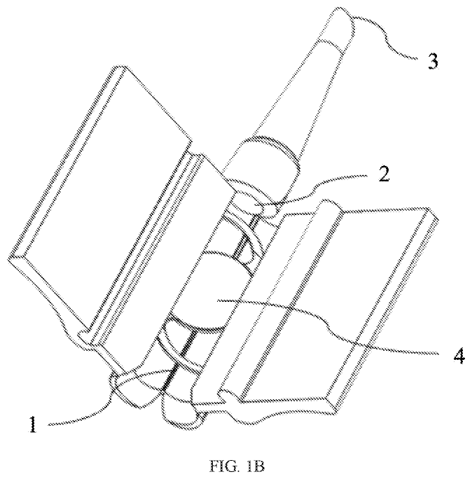

FIG. 1A shows the traditional butterfly-type IOL cartridge and FIG. 1B shows a pre-loaded IOL cartridge. Intraocular lenses are loaded onto area 1 when in use. The area is coated via layer-by-layer coating of polymer solutions to facilitate the IOL delivery.

The components shown in the figure are each given reference numbers as follows:
1. IOL loading area
2. Feed orifice
3. Outlet orifice
4. Intraocular lens

The coating in the present invention is used as a lubricant on the inner surface of the intraocular lens cartridges, and it is a layer-by-layer coated polymer-based material which facilitates the implantation of intraocular lenses (4). The subject matter of the invention, in relation to the prior art, enables to develop a lubricious coating which will enable the intraocular lens (IOL) (4) to be easily implanted through the cartridge without damaging it, remains stable during its long shelf-life.

In the present invention, multilayer polymer coatings are obtained via LBL deposition of positively charged polymers and negatively charged polymers in a spin coating device. Prior to the coating, the cartridges are treated with plasma for improved wettability. The cartridges are first coated with positively charged polymers. Next, the cartridges are coated with negatively charged polymers. This cycle makes one bilayer and repeated to desired numbers of bilayers which is preferably 5 bilayers.

The present invention, a spin-assisted layer-by-layer polymer coating for intraocular lens (IOL) cartridge including traditional butterfly and pre-loaded types, is developed in order to facilitate implantation of intraocular lenses (4), to enable the implantation of intraocular lens (4) through the cartridge easily without damaging it, to enable it to be stable, biocompatible and lubricious during its long shelf life and for those purposes, it comprises at least one bilayer that includes at least one positively charged polymer and at least one negatively charged polymer.

In the present invention, the coating components are selected from positively charged polymers and negatively charged polymers. The positively charged polymer solutions are prepared in deionized water and because they are water soluble polymers, it is essential that ratio of deionized water is at least 50% and the residual part of 50% can be polar organic solvents or their mixtures which have lower boiling point than water to vaporize rapidly while the coating obtain. The negatively charged polymer solutions are prepared in deionized water/ethanol mixture. In the spin-assisted layer-by-layer polymer coating, weight of positively charged polymer is 0.1-3.0% and weight of negatively charged polymer is 0.1-10.0%.

One of the coating components includes a positively charged polymers, but are not limited to, those selected from a group that comprises polyethyleneimine, polydiallyldimethylammonium chloride, polyallylamine hydrochloride, polyvinylamine, polyquaternium-7, polyquaternium-10, polyquaternium-24, polyquaternium-39, polyquaternium-44, chitosan, polylysine, poly(2-(dimethylamino)ethyl methacrylate), and their analogues and derivatives and any mixtures thereof.

One of the coating components includes a negatively charged polymers, but are not limited to, those selected from a group that comprises carboxymetyl cellulose, sodium hyaluronate, sodium poly(styrene sulfonate), polyether polyurethane, poly(acrylic acid), poly(p-styrene carboxylic acid), polyvinyl sulfonic acid, polygalacturonic acid, poly(methacrylic acid), sodium alginate, and their analogues and derivatives and any mixtures thereof.

The material of the cartridge that is coated with the layer-by-layer deposition of oppositely charged polymers is polyolefin such as polypropylene.

In one of the embodiments of the present invention, the outlet orifice (3) diameter of intraocular lens cartridge is minimum 1.0 mm, maximum 3.0 mm.

In the present invention, a production method of spin-assisted layer-by-layer polymer coating for intraocular lens cartridge comprises the following steps:
i. Applying plasma treatment to cartridges for 1-30 minutes at power of 10-100 W,
ii. Spin coating of positively charged polymer on the inner surface of the cartridge,
iii. Then, spin coating of negatively charged polymer on the positively charged polymer coating on the inner surface of the cartridge,
iv. Repeating the cycle which includes steps (ii) and (iii) to obtain two or more bilayers,
v. Drying the coating surfaces.

In one of the embodiments of the present invention, while spin coating process, spin rotation speed is 100-2500 rpm and its time is 3-60 seconds.

In one of the embodiments of the present invention, drying process is carried out on the coating surfaces at a temperature of 50-90° C. for 5-120 minutes.

In the process steps of coating positively charged polymer and coating negatively charged polymer on the inner surface of cartridge, the polymer is applied on the inner surface of cartridge by 10-200 μl volume. The cartridge placed in the spin coating device is rotated to homogeneously distribute the polymer solution and to remove excess solution following the process steps of coating positively charged polymer and coating negatively charged polymer on the inner surface of cartridge.

Although the coating components preferably do not pass into the eye or to the intraocular lens (4) during use, i.e., during the surgical procedure, coating components that are substantially non-irritating to ocular tissue and/or are substantially biocompatible with ocular tissue are particularly useful. The coating components provide the enhanced lubricity of an interior surface of the IOL cartridge through which the IOL (4) travels as it is being delivered. Such coating components are preferably effective to provide such enhanced lubricity for relatively long periods of time, for example, for 12 months to 60 months. Accordingly, the traditional cartridge injector and preloaded systems have a relatively long shelf life and can be used after being packaged, sterilized and stored for relatively long periods of time and still possess the commercial advantages of enhanced lubricity and stability of the coating, i.e., with no transfer of the coating component to the surface of the IOL (4) during storage or during delivery of the IOL (4).

In other words, the layer-by-layer coated cartridges can withstand ethylene oxide sterilization and provide excellent adhesion to the cartridge thereby inhibiting or minimizing the transfer of the coating into the eye or onto the intraocular lens (4) during the delivery of the intraocular lens (4) in surgical implantation.

### EXAMPLES

### Example 1

Multilayer polymer coatings were obtained via spin-assisted LBL deposition of positively charged polymers and negatively charged polymers in a spin coating device. The coating solutions were prepared in aqua and/or water/ethanol (EtOH) mixtures and their compositions and coating parameters were shown in Table 1. In the present invention, both butterfly-type cartridges and the cartridges using in preloaded injector systems were used to evaluate the performance of IOL (4) delivery tests. Prior to the coating, both types of cartridges, which are manufactured by VSY Biotechnology, were exposed to plasma treatment for improved wettability. The plasma treated cartridges were placed to the spin coating device. The cartridges were first spin-coated with positively charged polymer solution. Next, the cartridges were spin-coated with negatively charged polymer solution. This cycle makes one bilayer. The cycle is repeated to reach the desired number bilayers. The coated cartridges were dried in an oven and were then subjected to ethylene oxide sterilization.

TABLE 1
Coating Parameters

| Parameters | Value |
|---|---|
| Concentration of positively charged polymer solutions (%) | 0.1-3 |
| Concentration of negatively charged polymer solutions (%) | 0.1-10 |
| Volume of the coating solution (μL)* | 10-200 |
| Spin rotation speed (rpm) | 100-2500 |
| Spin time (sec) | 3-60 |

*These are the volumes for both positively and negatively charged polymer solutions separately.

IOL (4) delivery tests of coated cartridges were performed after ethylene oxide sterilization. In these tests, mid-power acrylic hydrophilic (20.5 D, Acriva UD 613, VSY Biotechnology) and acrylic hydrophobic (21 D, Enova GF3, VSY Biotechnology) IOLs (4) were used. Ophthalmic viscoelastic gels (Protectalon 1.4%, VSY Biotechnology) were used for butterfly-type cartridge injector systems, whereas balanced salt solutions (BSS) were used for preloaded cartridge injector systems in the IOL (4) delivery tests.

The IOL (4) delivery tests for butterfly-type cartridge injector systems (outlet orifice (3) diameter of 2.2 mm) were performed with the following process steps:
1. Applying viscoelastic gel into the cartridge
2. Placing the intraocular lens (4) in the IOL loading area (1)
3. Assembling the cartridge into the injector system
4. Performing IOL (4) delivery tests
5. Surface controlling of IOLs (4) under optical microscope

The IOL (4) delivery tests for preloaded cartridge injector systems were performed with the following process steps:
1. Placing the intraocular lens (4) in the IOL loading area (1)
2. Assembling the cartridge into the injector system
3. The BSS penetrating into the areas on the inner part of the IOL (4) and cartridge
4. Performing IOL (4) delivery tests
5. Surface controlling of IOLs (4) under optical microscope

The average of the ten injection force measurements was calculated, and their results were summarized in Table 2 and 3. The coated cartridges (one to five bilayers) were obtained with excellent lubricity and all IOLs (4) were without damage in IOL (4) delivery tests. The injection force data reported in Table 2 and 3 indicates that five bilayer coating required the least amount of injection force to deliver an IOL (4). Besides, there was not observed the residual coating material that transferred from inner surface of the cartridge onto the surface of the IOLs (4).

TABLE 2
IOL (4) delivery test performance of butterfly-type cartridge injector systems

| Bilayer | Lens | Injection | Coating | |
|---|---|---|---|---|
| number (n) | Delivery | Force (N) | Transfer | IOL Damage |
| 1 | passed* | 15.8N | no | no scratch and tear |
| 2 | passed* | 13.1N | no | no scratch and tear |
| 3 | passed* | 12.6N | no | no scratch and tear |
| 4 | passed* | 10.4N | no | no scratch and tear |
| 5 | passed* | 9.9N | no | no scratch and tear |
| 1 | passed** | 14.2N | no | no scratch and tear |
| 2 | passed** | 13.4N | no | no scratch and tear |
| 3 | passed** | 11.8N | no | no scratch and tear |
| 4 | passed** | 10.4N | no | no scratch and tear |
| 5 | passed** | 9.7N | no | no scratch and tear |

*Mid power (20.5 D) Acriva UD 613 IOLs (4) and Protectalon 1.4% viscoelastic were used for testing lens deliveries.
**Mid power (21 D) Enova GF3 IOLs (4) and Protectalon 1.4% viscoelastic were used for testing lens deliveries.

TABLE 3
IOL (4) delivery test performance of preloaded cartridge injector systems

| Bilayer | Lens | Injection | Coating | |
|---|---|---|---|---|
| number (n) | Delivery | Force (N) | Transfer | IOL Damage |
| 1 | passed* | 10.9N | no | no scratch and tear |
| 2 | passed* | 10.1N | no | no scratch and tear |
| 3 | passed* | 8.2N | no | no scratch and tear |
| 4 | passed* | 7.5N | no | no scratch and tear |
| 5 | passed* | 7.1N | no | no scratch and tear |

*Mid power (21 D) Enova GF3 IOLs (4) and BSS were used for testing lens deliveries.

### Example 2

Accelerated aging study was performed to estimate the shelf-life of LBL-coated cartridges stored at 44° C. over 489 days. This simulates at least 5 year of room temperature performance based on the Arrhenius' equation:

$$Accelerated Aging Duration = \frac{Real Time Duration}{{Q}_{10}^{\frac{{T}_{AA} - {T}_{RT}}{10}}}$$

$${T}_{AA} = 44 ° C .$$

$${T}_{RT} = 25 ° C .$$

$${Q}_{10} = 2 ,$$

where T_{AA} is the accelerated temperature, T_{RT} is ambient temperature and Q₁₀ is aging factor. Common Q₁₀ (aging factor) is 2 for medical devices.

The cartridges having one-bilayer coating and mid power (20.5 D) Acriva UD 613 and mid power (21 D) Enova GF3 IOLs were used for accelerated aging study. The cartridges were tested at day 98 (1 year RT), day 196 (2 years RT), day 293 (3 years RT), day 391 (4 years RT) and day 489 (5 years RT). After each specific time, IOL (4) delivery and cytotoxicity tests were performed. The results had shown equal excellent lubricity and the coated cartridges were not cytotoxic before and after aging. The average of the ten injection force measurements was calculated, and their results were summarized in Table 4. All IOLs (4) were without damage in IOL (4) delivery tests. Besides, there was not observed the residual coating material that transferred from inner surface of the cartridge onto the surface of the IOLs (4).

TABLE 4
Accelerating aging study performance of butterfly-type cartridge
injector systems

| | Lens | Injection | Coating | |
|---|---|---|---|---|
| Time (day) | Delivery | Force (N) | Transfer | IOL Damage |
| 98 | passed* | 16.1N | no | no scratch and tear |
| 196 | passed* | 17.1N | no | no scratch and tear |
| 293 | passed* | 17.5N | no | no scratch and tear |
| 391 | passed* | 18.9N | no | no scratch and tear |
| 489 | passed* | 18.4N | no | no scratch and tear |
| 98 | passed** | 15.1N | no | no scratch and tear |
| 196 | passed** | 14.6N | no | no scratch and tear |
| 293 | passed** | 15.7N | no | no scratch and tear |
| 391 | passed** | 15.9N | no | no scratch and tear |
| 489 | passed** | 15.8N | no | no scratch and tear |

*Mid power (20.5 D) Acriva UD 613 IOLs (4) and Protectalon 1.4% viscoelastic were used for testing lens deliveries.
**Mid power (21 D) Enova GF3 IOLs (4) and Protectalon 1.4% viscoelastic were used for testing lens deliveries.

TABLE 5
Accelerating aging study performance of preloaded cartridge injector systems

| | Lens | Injection | Coating | |
|---|---|---|---|---|
| Time (day) | Delivery | Force (N) | Transfer | IOL Damage |
| 98 | passed* | 11.1N | no | no scratch and tear |
| 196 | passed* | 11.2N | no | no scratch and tear |
| 293 | passed* | 12.7N | no | no scratch and tear |
| 391 | passed* | 12.5N | no | no scratch and tear |
| 489 | passed* | 12.9N | no | no scratch and tear |

*Mid power (21 D) Enova GF3 IOLs (4) and BSS were used for testing lens deliveries.

In summary, the LBL-coated cartridges of the present invention are capable of delivering a foldable IOL (4) with minimum injection force, without IOL (4) damage, into the eye through a smaller incision. The inner surface of the coated cartridges is highly hydrophilic and lubricious when wet with BSS or viscoelastic solution. The multilayer polymer coatings do not detach from the cartridge surface and can withstand ethylene oxide sterilization. Thus, eliminating the coating transfer into the eye during the IOL (4) insertion process. The LBL coatings can also be used to design a preloaded device containing hydrophobic IOL (4) packaged in a dry state.

The advantages of the coating obtained within the scope of the invention before the state of the art can be listed as follows:
It can be used with silicone-based, hydrophobic and hydrophilic acrylic IOLs (4).
It can be used with viscoelastic solutions, balanced salt and saline solution during IOL (4) implantation.
It can be used with both traditional butterfly type and preloaded cartridge injector systems.
It can be used to design a preloaded device containing hydrophobic IOL (4) packaged in a dry state.
Having a stable, sterile and biocompatible coating for a long shelf-life (5 years).
Time-saving coating process.
It provides the possibility of adjustable coating thickness by reaching the desired number of layers.

1. A spin-assisted layer-by-layer polymer coating for intraocular lens cartridge including traditional butterfly and pre-loaded types; which is developed in order to facilitate implantation of intraocular lenses, to enable the implantation of intraocular lens through the cartridge easily without damaging it, to enable it to be stable, biocompatible and lubricious during its long shelf life; comprising at least one bilayer that includes:
at least one positively charged polymer, and
at least one negatively charged polymer.

2. The spin-assisted layer-by-layer polymer coating for intraocular lens cartridge according to claim 1, wherein the positively charged polymer is selected from a group comprised of polyethyleneimine, polydiallyldimethylammonium chloride, polyallylamine hydrochloride, polyvinylamine, polyquaternium-7, polyquaternium-10, polyquaternium-24, polyquaternium-39, polyquaternium-44, chitosan, polylysine, poly(2-(dimethylamino)ethyl methacrylate), and their analogues and derivatives and mixtures thereof.

3. The spin-assisted layer-by-layer polymer coating for intraocular lens cartridge according to claim 1, wherein the negatively charged polymer is selected from a group comprised of carboxymetyl cellulose, sodium hyaluronate, sodium poly(styrene sulfonate), polyether polyurethane, poly(acrylic acid), poly(p-styrene carboxylic acid), polyvinyl sulfonic acid, polygalacturonic acid, poly(methacrylic acid), sodium alginate, and their analogues and derivatives and mixtures thereof.

4. The spin-assisted layer-by-layer polymer coating for intraocular lens cartridge according to claim 1, wherein the positively charged polymer solutions are prepared in deionized water.

5. The spin-assisted layer-by-layer polymer coating for intraocular lens cartridge according to claim 1, wherein the negatively charged polymer solutions are prepared in deionized water/ethanol mixture.

6. The spin-assisted layer-by-layer polymer coating for intraocular lens cartridge according to claim 1, wherein the intraocular lenses are silicone-based, hydrophobic acrylic and hydrophilic acrylic intraocular lenses.

7. The spin-assisted layer-by-layer polymer coating for intraocular lens cartridge according to claim 1, wherein it is activated with viscoelastic solutions, balanced salt solution and saline solution during intraocular lens implantation.

8. The spin-assisted layer-by-layer polymer coating for intraocular lens cartridge according to claim 1, wherein the diameter of the outlet orifice intraocular lens cartridge is minimum 1.0 mm, maximum 3.0 mm.

9. The spin-assisted layer-by-layer polymer coating for intraocular lens cartridge according to claim 1, comprising 0.1-3.0% by weight of positively charged polymer and 0.1-10.0% by weight of negatively charged polymer.

10. A production method of spin-assisted layer-by-layer polymer coating for intraocular lens cartridge according to claim_1, further comprising:
i. applying plasma treatment to cartridges for 1-30 minutes at power of 10-100 W,
ii. spin coating of positively charged polymer on the inner surface of the cartridge,
iii. spin coating of negatively charged polymer on the positively charged polymer coating on the inner surface of the cartridge,
iv. repeating the cycle which includes steps (ii) and (iii) to obtain two or more bilayers, and
v. drying the coating surfaces.

11. The production method of spin-assisted layer-by-layer polymer coating for intraocular lens cartridge according to claim 10, wherein the polymer is applied on the inner surface of cartridge by 10-200 μl volume in the process steps of coating positively charged polymer and coating negatively charged polymer on the inner surface of cartridge.

12. The production method of spin-assisted layer-by-layer polymer coating for intraocular lens cartridge according to claim 10, wherein the cartridge placed in the spin coating device is rotated to homogeneously distribute the polymer solution and to remove excess solution following the process steps of coating positively charged polymer and coating negatively charged polymer on the inner surface of cartridge.

13. The production method of spin-assisted layer-by-layer polymer coating for intraocular lens cartridge according to claim 10, wherein spin rotation speed is 100-2500 rpm and its time is 3-60 seconds.

14. The production method of spin-assisted layer-by-layer polymer coating for intraocular lens cartridge according to claim 10, wherein the drying process is carried out on the coating surfaces at a temperature of 50-90° C. for 5-120 minutes.

15. The spin-assisted layer-by-layer polymer coating for intraocular lens cartridge according to claim 2, wherein the positively charged polymer solutions are prepared in deionized water.

16. The spin-assisted layer-by-layer polymer coating for intraocular lens cartridge according to claim 3, wherein the positively charged polymer solutions are prepared in deionized water.

17. The spin-assisted layer-by-layer polymer coating for intraocular lens cartridge according to claim 2, wherein the negatively charged polymer solutions are prepared in deionized water/ethanol mixture.

18. The spin-assisted layer-by-layer polymer coating for intraocular lens cartridge according to claim 3, wherein the negatively charged polymer solutions are prepared in deionized water/ethanol mixture.

19. The spin-assisted layer-by-layer polymer coating for intraocular lens cartridge according to claim 4, wherein the negatively charged polymer solutions are prepared in deionized water/ethanol mixture.

20. The spin-assisted layer-by-layer polymer coating for intraocular lens cartridge according to claim 2, wherein the intraocular lenses are silicone-based, hydrophobic acrylic and hydrophilic acrylic intraocular lenses.
